(19) United States
(12) Patent Application Publication
(10) Pub. No.: US 2002/0028467 A1
(43) Pub. Date: Mar. 7, 2002

(54) Dispensing of suspensions on a microscale for the preparation of material samples in combinatorial materials research, and the testing thereof

(76) Inventors: Hans-Josef Sterzel, Dannstadt-Schauernheim (DE); Klaus Kuhling, Mutterstadt (DE)

Correspondence Address: Herbert B. Keil, KEIL & WEINKAUF, 1101 Connecticut Ave., N.W., Washington, DC, 20036, US

(21) Appl. No.: 09/888,591

(22) Filed: Jun. 26, 2001

(30) Foreign Application Priority Data
Jun. 29, 2000 (DE) 10031587.9

(51) Int. Cl. G01N 33/53; B01J 23/00; G01N 33/557

(52) U.S. Cl. 435/7.1; 436/517; 502/300

(57) ABSTRACT

In a process for the combinatorial production of material samples in the form of a two-dimensional matrix in the surface region of a sheet-like substrate, at least two different dispensable material components are dispensed as a suspension from one or more dispensing devices which allow the release of individual suspension drops onto the same point of the substrate, so that materials of different composition are obtained in different surface regions of the substrate.

The present invention relates to a process for the combinatorial preparation of material samples in the form of a two-dimensional matrix in the surface region of a sheet-like substrate. The present invention also relates to a process for the combinatorial testing of samples obtained in this way.

The parallelized preparation and very fast serial or parallel testing of materials having suitable physical or chemical properties is an area of materials research which is currently increasing greatly in importance.

WO 98/47613 discloses a number of processes in which libraries of materials of potential interest can be generated by means of sputtering, CVD or PVD techniques. At its core, this application relates to the use of suitable mask techniques which facilitate defined deposition of at least two components (which exist as separate substrates) onto a single substrate, giving composite materials. Furthermore, complete libraries of materials of different composition can be generated using the process by the generation of gradients on the sputtered substrate.

The processes described have a number of disadvantages. Firstly, continuous compact libraries are generated on a substrate; it is only later through mechanical separation that these can be tested separately from one another for desired properties and analyzed with respect to their composition. Secondly, the sample amounts prepared by these substrate coating techniques are very small (a few milligrams or fractions thereof as thin layers on substrates with layer thicknesses in the nanometer to lower micron region), so that a defined treatment, such as sintering processes or treatment with certain media (liquids or gases), causes difficulties, in particular in the reproduction of process parameters or the reproducible dispensing of sample quantities. A further disadvantage of the sputtering process is that the morphology of the resultant materials may differ greatly with respect to crystallinity and particle size from that of materials produced by conventional methods, i.e. by sintering of powder mixtures. Success of this combinatorial method is thus uncertain since material properties, such as hardness, ion conductivity, thermal conductivity, dielectric constant, electrical conductivity, thermoelectric force, magnetic properties and porosity, are highly dependent, inter alia, on the crystallinity, the crystallographic nature of the crystallites or the particle sizes, the defects and grain boundaries and other parameters which can be severely affected by the production parameters, and on the starting powders.

DE-A 199 55 789, which has an earlier priority date than the present application, relates to a process for the combinatorial preparation of a library of materials in which at least two different sprayable material components are sprayed onto a substrate from at least two spray nozzles. In particular, spray pyrolysis is carried out here.

It is an object of the present invention to provide a process for the combinatorial production of material samples in the form of a two-dimensional matrix in the surface region of a sheet-like substrate which avoids the disadvantages of the known processes and allows a simplified process procedure. It is a further object of the present invention to provide a reliable design of the equipment in the process. The materials obtained should be prepared and analyzed under conditions as close to practice as possible in order that specific effects caused by the combinatorial production process are suppressed.

We have found that this object is achieved by a process for the combinatorial production of material samples in the form of a two-dimensional matrix in the surface region of a sheet-like substrate in which at least two different dispensable material components are dispensed as a suspension from one or more dispensing devices which allow the release of individual suspension drops onto the same point of the substrate, so that materials of different composition are obtained in different surface regions of the substrate.

The process according to the invention is particularly advantageously carried out using special dispersion aids and dispersion techniques as described below.

The sheet-like substrate is preferably arranged horizontally and is charged vertically from above, with the dispensing devices being arranged in such a way that the dispensing directions are in a plane perpendicular to the sheet-like substrate or angled thereto.

Any desired suitable dispensing devices which allow the release of individual suspension drops can be employed in accordance with the invention. The dispensing here is preferably carried out by displacing movable plungers in the dispensing device (or in a corresponding cavity of the dispensing device). The dispensing devices employed are particularly preferably syringes. In the particularly advantageous miniaturized embodiment of the process according to the invention, use is made of microliter syringes, which have a volume of from 1 to 1000 microliters, particularly preferably from 10 to 100 microliters.

A multiplicity of materials of different composition are preferably generated on a substrate. Preferably at least 100, particularly preferably at least 1000, different materials are preferably formed on the substrate.

The powders present as components of the material combinations to be prepared are preferably converted into highly concentrated particle suspensions using dispersion media and organic solvents and/or water. These suspensions are preferably sucked directly into a suitable syringe or cannula directly via solid plungers in the manner of microliter syringes or indirectly via barrier liquids serving as liquid plungers. The suspensions sucked in are then deposited at a preselected location in the surface region of a sheet-like substrate under computer control of the amount and speed.

The term “in the surface region” means that the materials are formed, for example, on a sheet-like substrate with the dispensed material component not penetrating into the substrate. This is the case, for example, in the case of smooth metal, ceramic or plastic substrates, in which, for example, the dispensed material components are accommodated in recesses.

However, they can also be at least partially porous substrates into which the dispensed material components adsorb or penetrate at least partially. In this case, the material is formed in accordance with the invention in the uppermost layer of the sheet-like substrate, i.e. in the surface region. In this case, the penetration depth of the dispensed material components is preferably a maximum of 100 μm, in particular a maximum of 10 μm.

The term “sheet-like substrate” means a substrate which extends to a significantly greater extent in two spatial directions than in the third spatial direction. The sheet-like substrate need not be planar, but can, for example, be a series of wells or recesses on or in a plate. For example, it can be a spot plate or a corresponding plate with recesses at regular separations. However, the precise design of the sheet-like substrate is not restricted. Individual sections (matrix elements) on the sheet-like substrate may also be separated off in another suitable manner.

The term “different surface regions” describes regions on the surface of the substrate which are spatially different from one another. These regions are preferably spatially delimited from one another, so that the sheet-like substrate is divided into individual defined positions.

According to one embodiment of the invention, the sheet-like substrate is divided into individual defined positions through the dispensing operations taking place on the substrate at a spatial separation which prevents dispensed suspension drops coalescing on the substrate.

According to a further embodiment of the invention, the sheet-like substrate is divided into individual defined positions through the sheet-like substrate being placed on a matrix plate provided with holes, the dispensing taking place into the holes, the dispensed suspension drops being dried at least to the extent they do not coalesce on the substrate when the matrix plate is lifted off, and the matrix plate being lifted off after the drying.

A total of from 1 to 1000 μl particularly preferably from 5 to 100 μl, of suspension are preferably dispensed per position on the substrate.

In a preferred procedure, the actual substrate is laid on a matrix plate provided with holes. The suspensions are dispensed simultaneously or successively into the holes, the suspensions are mixed via a small suitable mixing device, and the matrix plate is lifted off after partial drying of the mixture. Materials employed for the matrix plate are fluorinated plastics of low wettability, such as polytetrafluoroethylene, polyvinylidene fluoride or ECTFE. Depending on the screening requirements, the matrix plates have thicknesses of from 0.5 to 5 mm, preferably from 1 to 2 mm. The holes located therein for accommodating the suspensions or mixtures thereof have separations of 1-2 mm, and their diameter at the base is 1-3 mm. For better ejection, the holes have inclines of at least 5°, causing the material samples to attain the shape of truncated cones which stand on the substrate plate by means of the larger surface. In this way, from 1000 to 2000 material samples can be accommodated on a substrate plate with an edge length of 150×150 mm. It is also possible to work without matrix plates. To this end, it must be ensured that drops deposited on the substrate plate, for example from microliter syringes, do not flow apart. In order to ensure this, the solid particles are suspended in liquids which, on the one hand, have the highest possible surface tension, and on the other hand do not form hydrogen bridges with the substrate plate. Thus, a drop of formamide with its high surface tension of 58 dyn/cm (room temperature) hardly flows apart at all on a fluorinated plastic plate, but does so to a greater extent on a silicon carbide plate. By contrast, solvents of high surface tension which are virtually unable to form hydrogen bridges, in particular propylene carbonate, ethylene carbonate or gamma-butyrolactone, which form drops with contact angles of virtually 90° on silicon carbide, titanium nitride or gold, have an ideal behavior.

The invention also relates to a process for the combinatorial testing of the material libraries obtained in this way in which the material samples present on individual defined positions of the substrate are analyzed for a desired property by physical and/or chemical methods.

The basic idea of the invention using two or more particle suspensions consists in that different material components are applied to a substrate with defined sample positions by means of two or more dispensing devices.

In one embodiment, the suspensions of the components are drawn up separately for each component from a stirred stock vessel by means of microliter syringes moved by actuators. The microliter syringes are moved toward one another, each on a robot arm, so that they lie on the envelope of an imaginary cone and the openings of the channels touch at virtually a single point. At this point, defined amounts of the respective suspensions are dispensed from syringes via the actuators, and the drop generated in this way is deposited on the substrate plate either by lowering the syringe or raising the substrate plate until the drop touches the substrate plate, and lowering the syringe. The deposition either takes place freely on the plate or into the cavity of a matrix plate.

If simultaneous dispensing from the syringes is carried out at different rates, additional mixing is unnecessary for total volumes of about 10 μl; the diffusion rate in the solid is often so great in the subsequent sintering step that the components are distributed in the solid in accordance with the thermodynamic equilibrium.

Otherwise, the component mixture can be mixed within a cavity of a matrix plate. This can be carried out in the form of premixing with subsequent placing of the mixture on the sheet-like substrate or directly thereon or in cavities thereof.

In a preferred embodiment, the drops of the components are not deposited immediately on the actual substrate plates, but are deposited on a further plate, the components are mixed on the separate plate, and the mixture is then transferred onto the actual substrate plate by means of a single dispensing syringe. This has the advantage that a homogeneously mixed material is reliably obtained. In addition, a somewhat larger amount can be mixed, for example 20-100 μl, which can be dispensed with a smaller error, and only some of this amount, for example 5 to 20 μl, is used. In this procedure, the dispensable material components are firstly dispensed and mixed on an auxiliary substrate, and the mixture is then taken up by the dispensing device and dispensed onto the substrate.

It is also possible to avoid direct contact of a syringe plunger with the suspensions by moving only an inert liquid which is immiscible with the suspension medium over the syringes themselves, and sucking up and dispensing the suspensions by means of the liquid plunger generated in this way. This procedure provides the advantage that a metallic plunger is not in contact with suspended hard particles.

In a further embodiment, it is possible to suck the suspensions of the components successively into the same syringe using a solid or liquid plunger, and to dispense the suspensions therefrom, with the syringe being completely emptied. In this embodiment, subsequent homogenization of the component mixture is advisable.

It is advisable to flatten the dome of the samples after drying and so long as the powder accumulation is still flowable, via an automatically guided pin with a planar end face, in order to have the flattest possible contact surface for measurement probes during later screening. This is of less importance if use is made of annular probes, which lie on the camber.

The filling of the syringes and dispensing of the suspensions is controlled via a computer, which also controls the screening apparatus. In accordance with specifications, the amounts of the individual components are assigned to the local coordinates on the substrate plate and dispensed or premixed there. The mixing ratios here can be determined via a random-number generator. Specifications to be entered are, for example, the proportioning of the individual components, or it is specified that certain components should be present in the mixture in a minimum or maximum proportion.

The combinations carried out, which are normally not repeated, are stored. If it is noted at the position of a spray actuator that the syringe needs refilling, all syringes move to the storage containers and are refilled.

For the preparation and deposition of a component mixture, only 1-100 seconds are required per mixture.

In the same time scale, the samples are screened. Including the sintering to give the actual material, which follows the synthesis, and parallel use of the times of sample adjustment, sintering and screening, from 1000 to 5000 material samples can be screened per 24 hours, depending on the sample size and test duration.

After partial drying of the material samples on the substrate plate, the substrate plate is transferred into a sintering furnace, in which the actual materials to be screened are generated at temperatures and under atmospheres which are adequate for the material. The samples are heated here at rates of from 1 to 20° C./min, left at the sintering temperature for from 0.5 to 5 hours and then cooled in the furnace. During sintering, the samples usually shrink corresponding to the volume proportion which was not filled with inorganic powder. At a volume proportion of 50%, the linear shrinkage is, for example, about 21%. After removal of the plate from the furnace and after cooling, it is transferred to the screening robot, which tests the property to be optimized.

The screening robot moves its probe over the samples in accordance with the computer program and the coordinates of the material samples and tests a cycle corresponding to the test specifications. The test probes are designed in accordance with the properties to be tested: if mechanical properties, such as hardness or rigidity, are to be tested, a rigid substrate plate is used, for example made of silicone carbide. The test probe carries a diamond, which is pressed against the samples, the force/path diagram is measured and stored in the computer sample by sample. The measurements can be carried out at various temperatures or with a temperature-controlled probe.

The screening of dielectric properties is carried out by placing the material samples on an electrically conductive substrate plate, for example silicon carbide or a gold- or titanium nitride-coated ceramic or metal plate, and applying the probe at constant test force. An alternating voltage of interesting frequency and voltage is then applied to the material sample, and the dielectric properties of interest are measured. This too can be carried out at various measurement temperatures.

For the screening of magnetic properties, the probe carries a minicoil to which currents of variable frequency are applied. The information on magnetic permeability, hysteresis values, quality factors or other important properties are obtained via a secondary coil. In order to avoid interfering eddy currents, the substrate plate should not be made of a non-conductive material such as aluminum oxide or zirconium oxide.

For the screening of the oxidation or scale resistance of materials, the samples including inert substrate plates are heated in the atmosphere of interest, and the disappearance of the reflection capacity, the formation of annealing colors or other discolorations and changes are observed using a camera.

For the screening of media resistance, the material samples including inert substrate plate made of ceramic materials are exposed to the gaseous or liquid media at temperatures of interest and, in a first step, tested for optically visible changes.

Electrical resistances or impedances can be tested as a function of frequency via an applied electrode.

Thermal stresses and thermocurrents can be screened relative to one another on the individual material samples by applying a temperature-controlled electrode.

The ion conductivity of the material samples can likewise easily be screened. For example, the conduction capacity for oxygen ions can be screened by measuring the electrical resistance at certain temperatures and oxygen partial pressures.

The ability of material samples to store gases, for example hydrogen or oxygen, can be screened rapidly by exposing the substrate plate with the material samples to the gas to be tested at room temperature. The samples are subsequently brought into brief contact with a heated tube which is connected to a mass spectrometer in order to measure whether and to what extent the gas is released at prespecified temperatures or during heating.

Opto-electrical effects can be screened rapidly by measuring the electrical resistance or formation of a photovoltage as the exposure is changed.

Screening for supraconductivity is particularly simple. The material samples are sheared off the substrate plate and transferred into a thermos flask containing, for example, liquid nitrogen. A permanent magnet is located at the bottom of the flask. Material samples which float or are suspended have critical temperatures of at least −196° C. The level of the critical temperature limit to be screened can be set depending on the cooling medium.

Many market-relevant material properties can thus be screened quickly and simply.

If tests give measurements directly or via measurement converters, these are stored for each material sample or coordinate on the substrate plate, for example as measurement curves. The computer program is designed in such a way that a particularly characteristic value in a color scale is displayed on a screen. Thus, an overview of these characteristic quantities over the entire substrate plate is obtained at a glance. Points which appear interesting are clicked (called up), and both the measurement curve and further important parameters, such as dispensed composition, coordinates, etc., are obtained. The material components are preferably selected from suspensions and powders of the elements from groups IB, IIB, IIIB, IVB, VB, VIB, VIIB and VIII, lanthanoids, actinoids, IA, IIA, IIIA, IVA, VA and VIA or compounds or mixtures thereof. The term powder is taken to mean both metal powders and oxides, carbides or nitrides. The suspension media can be, for example, water, paraffins or mixtures of solvents with suitable dispersants, for example organic additives. If suspensions are dispensed, the particles present in the dispersion/suspension should preferably be smaller than 50 microns, particularly preferably smaller than 10 microns, in order to avoid blockage of the nozzles.

It is advantageous for the process according to the invention to convert finely divided powders into stable suspensions. In order to have to evaporate as little dispersion medium as possible and to obtain a high sintering density, the highest possible proportions of solid by volume should be employed at the same time as low viscosity of the suspensions to be dispensed. Volume contents of preferably from 20 to 50% by volume, particularly preferably from 35 to 45% by volume, are obtained on use of suitable dispersants.

Since the requirements of dispersants vary greatly due to the large number of different powders and possible dispersion media, uneconomical individual solutions for dispersion problems, i.e. a certain dispersant for a specific combination of powder and dispersion medium, are widespread.

By contrast, dispersants of the formula I

(R¹)ₓ-A-[(B-O)ₙ-Z], (I)

have a very broad spectrum of action.

In detail, the variables and groups have the following meaning:

A is oxygen or a —CO—O— group, preferably oxygen, if x and y are 1; furthermore nitrogen if x+y=3,

B is ethylidene or 1,2-propylidene, preferably ethylidene,

Z is one of the groups

[chemical structure]

where the following groups are preferred:

—CH₂CH₂N⁺R²R³—CH₂—CO₂—,

D⁻ is acetate, formate, propionate or hydroxide, preferably acetate,

E⁻ is carboxylate or sulfonate, preferably carboxylate,

M is a bridging group for completion of a pyrrolidone, succinimide or maleimide ring as the group

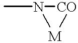

n is from 1 to 50, preferably from 2 to 25,

q is from 1 to 4, preferably 1,

x and y are 1 or 2, with the proviso that x+y is ≦3,

R¹ is a (C₈-C₃₀)-alkyl group or, if A=0, a (C₄-C₁₂)-alkyl-substituted phenyl radical, preferably a (C₁₂-C₁₈)-alkyl group, of these particularly mixtures of (C₁₃/C₁₅)-oxo alcohol radicals and (C₁₂/C₁₄)- and (C₁₆/C₁₈)-fatty alcohol radicals; in the case where A is oxygen, preferably tert-butyl, isooctyl- and isononylphenyl,

R², R³ and R⁴ are hydrogen, methyl or ethyl, preferably hydrogen or methyl, and

R⁵ is hydrogen or methyl.

The compounds (I) are alkylene oxide or polyalkylene oxide derivatives containing (R¹)ₓ-A- and -Z radicals as terminating groups.

The dispersants of the general formula (I) are used in accordance with the invention for the dispersion of finely divided solids in a flowable medium (dispersion medium). They are described, for example, in EP-A-0 582 209.

Solids of this type are preferably oxidic or nonoxidic inorganic powders and metal powders.

Finely divided means that the mean particle sizes are generally from 0.1 to 10 μm.

Flowable media (dispersion media) cover liquids which are liquid at room temperature, such as water or organic solvents, such as alcohols, ethers, ether alcohols, esters, ketones, aliphatic and aromatic hydrocarbons, halogenated hydrocarbons, amines, amides and nitro compounds. Mixtures of these substances can also be used.

Suspensions to be applied are obtainable in accordance with the invention from the above-mentioned finely divided solids, the flowable media and compounds of the formula (I).

The suspensions employed in accordance with the invention are prepared in a manner known per se. Thus, it has proven particularly successful to carry out the dispersion using ultrasound waves by adding the solid to the dispersion medium and dispersant in an ultrasound bath.

For larger batches, grinding, for example in a vibratory mill or ball mill, will advantageously be used. Extremely high-speed stirrers are also suitable for preparing the dispersion. These have speeds of from 3000 to 40,000 min⁻¹.

For the purposes of the invention, suitable highly effective dispersants for the preparation of stable, highly concentrated and low-viscosity suspension preparations also include dispersants of the following formulae:

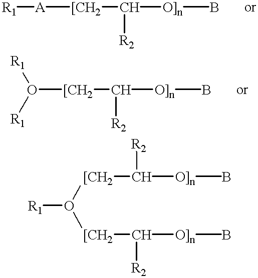

where R₁ is an aliphatic hydrocarbon radical having 10 to 40 carbon atoms, A is the —O— group or

—C(O)—O—,

R₂, independently of one another at different positions, are —H, —CH₃ or —CH₂—CH₃, B is the group —HSO₃, —H₂PO₄,

—(CH₂)ₓ—C(O)—OZ,

—(CH₂)—NH₂, —NH—(CH₂)ₓ—NH₂ or

—(CH₂)ₓ—C(O)—NY₂,

where Z is hydrogen, alkali metal or ammonium,

x is an integer from 1 to 3, and y is H or —CH₃,

D is the group >N— or

>N—C(O)—(CH₂)ₓ—,

where x is likewise as defined above, and n is an integer from 1 to 40.

From 0.1 to 10% by weight, particularly preferably from 0.5 to 5% by weight, based on the solid, of the dispersants are preferably employed.

If water or highly polar organic solvents are employed as suspension medium, so-called ionic stabilization of the particles to be kept in suspension has proven favorable. Dispersants which are salts of oleic acid with tetramethylammonium hydroxide or tetrabutylammonium hydroxide in the molar ratio 1:1 have proven particularly suitable for the application according to the invention.

The invention also relates to an apparatus for carrying out the process described, comprising a robot arm which can be moved under computer control and which carries one or more dispensing devices, for example syringes, with a capacity of from 1 to 1000 μl, and a computer for controlling the robot arm.

### EXAMPLES

The following examples illustrate the suspension preparation and the dispensability of the suspensions.

### Example 1

81.4 g of a non-porous iron carbonyl powder having particle sizes of from 2 to 8 μm, 11.8 g of t-amyl alcohol and 0.5 g of a dispersant prepared by ethoxylation of a C₁₃/C₁₅-fatty alcohol mixture with an average of 7 mol of ethylene oxide per mole of fatty alcohol, substitution of the terminal OH group by an NH₂ group and reaction thereof with maleic anhydride, were mixed for about 15 minutes at 15,000 min⁻¹ using an ultra-high-speed stirrer with a diameter of 12 mm.

A suspension was obtained which contained 40% by volume of iron powder.

This suspension was sucked into a microliter syringe with an overall capacity of 10 μl. The syringe plunger diameter was 0.5 mm, and the cannula diameter was about 0.2 mm with a cannula length of 45 mm.

2 μl were dispensed after the filled syringe had been stored for 5 minutes, 3 μl were dispensed after storage for a further 10 minutes and the remaining 5 μl were dispensed without difficulty after storage for a further 10 minutes. This dispensing procedure was repeated 10 times without difficulty.

### Example 2

39.5 g of an aluminum oxide powder with a peak of the particle size distribution at 0.9 μm, 13.8 g of propylene carbonate and 0.7 g of the tetrabutylammonium salt of oleic acid were mixed as in Example 1. A stable suspension was obtained which contained 45% by volume of aluminum oxide. It was possible to dispense this suspension without difficulties using a microliter syringe with the dimensions from Example 1 in the same dispensing cycle.

### Example 3

39.5 g of the aluminum oxide from Example 2 were mixed analogously to Example 2 with 11.5 g of water and 0.7 g of tetrabutylammonium salt of oleic acid. The stable suspension obtained had a content of 45% by volume of aluminum oxide and could be dispensed without difficulties as shown in Example 1.

### Example 4

330.5 g of the iron powder from Example 1 were mixed analogously to this example with 67.3 g of propylene carbonate and 2.0 g of the salt of oleic acid and tetrabutylammonium hydroxide. The resultant suspension had a content of 42% by volume of iron powder and could be dispensed without difficulties from a 10 μl syringe. Drops having a contact angle of 70-90° which did not flow apart were obtained on a wide variety of substrates, namely steel, aluminum oxide, gold and titanium nitride.

### Example 5

100 g of aluminum oxide from Example 2 were mixed analogously to this example with 31.4 g of gamma-butyrolactone and 3.0 g of the salt of oleic acid and tetrabutylammonium hydroxide. The resultant suspension had a content of 45% by volume of aluminum oxide powder and could be dispensed without difficulties from a 10 μl syringe.

The drops did not flow apart when deposited on steel, aluminum oxide, zirconium oxide, gold and titanium nitride. They had contact angles of 80-90°.

We claim:

1. A process for the combinatorial production of material samples in the form of a two-dimensional matrix in the surface region of a sheet-like substrate, in which at least two different dispensable material components are dispensed as a suspension from one or more dispensing devices which allow the release of individual suspension drops onto the same point of the substrate, so that materials of different composition are obtained in different surface regions of the substrate.

2. A process as claimed in claim 1, wherein the sheet-like substrate is arranged horizontally and is charged vertically from above, with the dispensing devices being arranged essentially in such a way that the dispensing directions are in a plane which is perpendicular to the sheet-like substrate or angled thereto.

3. A process as claimed in claim 1, wherein the dispensing is carried out by displacing movable plungers in the dispensing device.

4. A process as claimed in claim 1, wherein the dispensable material components are firstly dispensed and mixed on an auxiliary substrate, and the mixture is then taken up by the dispensing device and dispensed onto the substrate.

5. A process as claimed in claim 1, wherein the dispensable material components are selected from suspensions of powders of the elements from groups IB, IIB, IIIB, IVB, VB, VIB, VIIB and VIII, lanthanoids, actinoids, IA, IIA, IIIA, IVA, VA and VIA of the Periodic Table of the Elements, or compounds or mixtures thereof.

6. A process as claimed in claim 1, wherein the sheet-like substrate is divided into individual defined positions which are spatially delimited from one another.

7. A process as claimed in claim 6, wherein the sheet-like substrate is divided into individual defined positions through the dispensing taking place on the substrate at a spatial separation which prevents dispensed suspension drops coalescing on the substrate.

8. A process as claimed in claim 6, wherein the sheet-like substrate is divided into individual defined positions through the sheet-like substrate being laid on a matrix plate provided with holes, the dispensing taking place into the holes, the dispensed suspension drops being dried at least to the extent that they do not coalesce on the substrate when the matrix plate is lifted off, and the matrix plate being lifted off after this drying.

9. A process as claimed in claim 6, wherein a total of from 1 to 1000 μl of suspension are dispensed per position on the substrate.

10. A process for the combinatorial testing of material samples obtained by a process as claimed in claim 1, in which the material samples present at individual defined positions of the substrate are analyzed for a desired property using physical and/or chemical methods.

11. An apparatus for carrying out a process as claimed in claim 1, comprising a robot arm which can be moved under computer control and which carries one or more dispensing devices with a capacity of from 1 to 1000 μl, and a computer for controlling the robot arm.
